(19) United States
(12) Patent Application Publication
(10) Pub. No.: US 2021/0317681 A1
(43) Pub. Date: Oct. 14, 2021

(54) SYSTEM AND METHOD FOR A SELF-SANITIZING DOOR HANDLE

(71) Applicant: HACKA LABS, INC, SHERBORN, MA (US)

(72) Inventors: JEAN BERNARD KOEHL, SHERBORN, MA (US); BAYAN MASHREQUI, SHERBORN, MA (US); MARIE OLIVARES, SHERBORN, MA (US)

(73) Assignee: HACKA LABS, INC, SHERBORN, MA (US)

(21) Appl. No.: 17/224,413

(22) Filed: Apr. 7, 2021

Related U.S. Application Data
(60) Provisional application No. 63/007,159, filed on Apr. 8, 2020.

(51) Int. Cl. E05B 1/00; A61L 2/24; A61L 2/04; A61L 2/08; A61L 2/10

(52) U.S. Cl. CPC E05B 1/0069; A61L 2/24; A61L 2/04; A61L 2202/11; A61L 2/10; A61L 2/084; A61L 2202/14; A61L 2/085

(57) ABSTRACT

A system for a self-sanitizing door handle includes a door handle configured to operate opening and closing of a door, a sensor configured to detect presence of a person in the proximity of the door handle and contact of the door handle by the person, a device for sanitizing the door handle, and a processor configured to receive a signal from the sensor and activate the device for sanitizing the door handle after being contacted by the person.

### CROSS REFERENCE TO RELATED CO-PENDING APPLICATIONS

This application claims the benefit of U.S. provisional application Ser. No. 63/007,159 filed on Apr. 8, 2020 and entitled SYSTEM AND METHOD FOR A SELF-SANITIZING DOOR HANDLE, which is commonly assigned and the contents of which are expressly incorporated herein by reference.

### FIELD OF THE INVENTION

The present invention relates to a system and a method for a self-sanitizing a door handle, and more particularly to a door handle that is self-sanitized via a high intensity beam or heat.

### BACKGROUND OF THE INVENTION

Some of the most frequently contacted everyday items are door handles. As such, door handles tend to collect dirt and pathogens for various communicative diseases, such as, microbes, bacteria and viruses. Door handles for doors in public places are touched by many people and therefore are especially susceptible to accumulating these pathogens. Although many people practice good personal hygiene and wash their hands frequently, there are always some people that do not. Since both people that practice good personal hygiene and people that do not practice good hygiene touch door handles in public places, it is critical to sanitize the door handles after each use.

Traditional methods for cleaning door handles include soap, detergents, and chemicals applied during periodic cleanings of the door handles. However, these methods are not designed to be applied after each use of the door handle. Accordingly, there is a need for a method for sanitizing a door handle that can be applied after each use.

### SUMMARY OF THE INVENTION

The present invention relates to systems and methods for sanitizing, disinfecting and sterilizing door handles, and more particularly to door handles that are self-sanitized, disinfected and sterilized via a high intensity beam or heat.

In general, in one aspect, the invention features a system for a self-sanitizing door handle including a door handle configured to operate opening and closing of a door, a sensor configured to detect presence of a person in the proximity of the door handle and contact of the door handle by the person, a device for sanitizing the door handle, and a processor configured to receive a signal from the sensor and activate the device for sanitizing the door handle after being contacted by the person.

Implementations of this aspect of the invention may include one or more of the following features. The sensor may be one of infrared proximity sensors, capacitive proximity sensors, photoelectric proximity sensors or inductive proximity sensors. The device for sanitizing the door handle is configured to sanitize the door handle via high intensity electromagnetic radiation, or heat, or combination thereof. The device for sanitizing the door handle includes a laser and the laser emits a high intensity electromagnetic radiation laser beam that sanitizes an outer surface of the door handle. The high intensity electromagnetic radiation laser beam may be an infrared (IR) light, ultraviolet (UV) light, or visible light. The system further includes collimating and reflecting components for directing the laser beam onto the outer surface of the door handle. The device for sanitizing the door handle includes a heating source that generates a heat flash that sanitizes an outer surface of the door handle by applying heat. The outer surface of the door handle is heated to a temperature above 100° C. during sanitizing. The outer surface of the door handle includes a heat conductive coating. The heat conductive coating may be a metal, nickel, aluminum, chrome, tungsten, copper, silver, gold, zinc, alloys thereof, ceramic, silicon carbide, graphite or diamond. The system further includes a motor configured to rotate the door handle while being sanitized. The system further includes one or more warning indicators that emit one or more warning signals during the sanitizing of the door handle. The one or more warning indicators comprise light emitting diodes (LED). The system further includes a power source that provides power for operating the device for sanitizing the door handle, the sensor and processor. The system further includes one or more safety interlock devices that are configured to stop the sanitizing of the door handle when the door handle is touched by the person. The door handle may be an L-shaped door handle, a U-shaped door handle, or a knob-shaped door handle. The sensor, the sanitizing device and the processor are integrated within the door handle. The door handle may include a static internal component and a rotating outer component.

In general, in another aspect, the invention features a method for self-sanitizing a door handle including the following. First, providing a door handle configured to operate opening and closing of a door. Next, providing a sensor configured to detect presence of a person in the proximity of the door handle and contact of the door handle by the person. Next, providing a device for sanitizing the door handle. Next, providing a processor configured to receive a signal from the sensor and activate the device for sanitizing the door handle after being contacted by the person. Next, detecting the presence of the person in the proximity of the door handle and contact of the door handle by the person by the sensor and sending a first signal to the processor. Next, detecting removal of the person's contact of the door handle by the sensor and sending a second signal to the processor. Finally, activating the sanitizing device by the processor and self-sanitizing the door handle.

The details of one or more embodiments of the invention are set forth in the accompanying drawings and description below. Other features, objects, and advantages of the invention will be apparent from the following description of the preferred embodiments, the drawings and from the claims.

### BRIEF DESCRIPTION OF THE DRAWINGS

Referring to the figures, wherein like numerals represent like parts throughout the several views:

FIG. 1A is a front view of a door with an L-shaped door handle;

FIG. 1B is a front view of a door with a U-shaped bar door handle;

FIG. 1C is a front view of a door with knob door handle;

FIG. 2A is a front view of an L-shaped door handle that is self-sanitized via a laser beam, according to this invention;

FIG. 2B is a top view of the L-shaped door handle of FIG. 2A;

FIG. 3 is a transparent top view of the L-shaped door handle of FIG. 2A;

FIG. 4A is a side perspective view of the L-shaped door handle of FIG. 2A;

FIG. 4B is a side perspective view of the L-shaped door handle of FIG. 2A, during the self-sanitizing operation;

FIG. 5A is a front view of a U-shaped door handle that is self-sanitized via a laser beam, according to this invention;

FIG. 5B is a side view of the U-shaped door handle of FIG. 5A;

FIG. 6A is a transparent side view of the U-shaped door handle of FIG. 5A;

FIG. 6B is an enlarged view of Area A in the U-shaped door handle of FIG. 6A;

FIG. 7A is a side perspective view of the U-shaped door handle of FIG. 5A;

FIG. 7B is a side perspective view of the U-shaped door handle of FIG. 5A, during the self-sanitizing operation;

FIG. 8A is a front view of an L-shaped door handle that is self-sanitized via heat according to this invention;

FIG. 8B is a top view of the L-shaped door handle of FIG. 8A;

FIG. 9A is a transparent top view of L-shaped door handle of FIG. 8A;

FIG. 9B is an enlarged side view of L-shaped door handle of FIG. 8A;

FIG. 10A is a front view of a U-shaped door handle that is self-sanitized via heat, according to this invention;

FIG. 10B is a side view of the U-shaped door handle of FIG. 10A;

FIG. 11 is an enlarged side view of the U-shaped door handle of FIG. 10A;

FIG. 12A is a front view of knob door handle that is self-sanitized via heat, according to this invention;

FIG. 12B is a side view of the knob door handle of FIG. 12A;

FIG. 13A is a transparent side view of the knob door handle of FIG. 12A;

FIG. 13B is a cross-sectional view of the knob door handle of FIG. 12A.

### DETAILED DESCRIPTION OF THE INVENTION

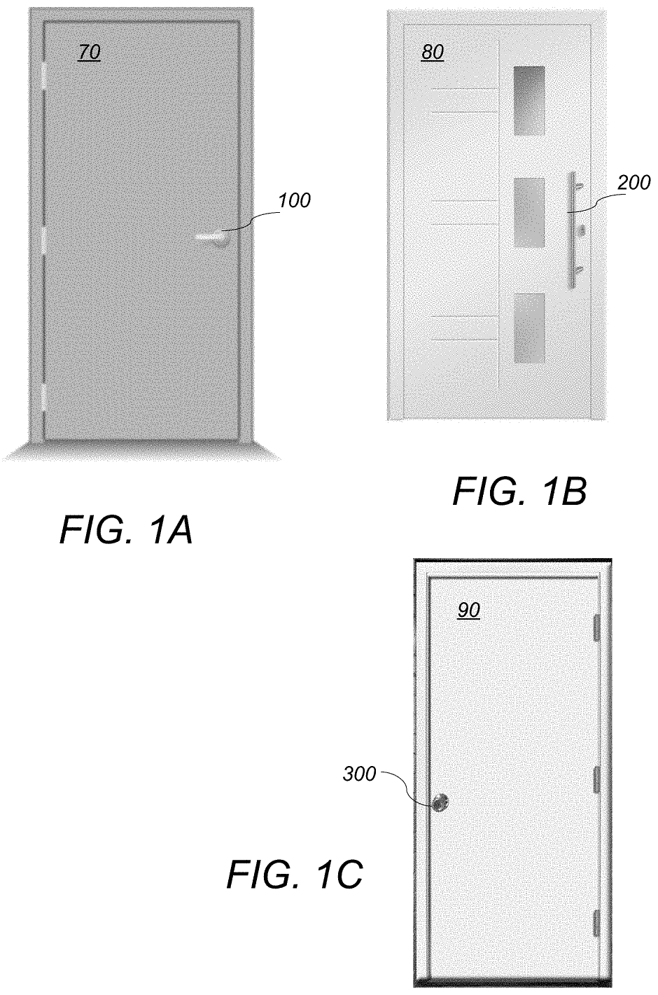

The present invention relates to systems and methods for sanitizing, disinfecting and sterilizing door handles, and more particularly to door handles that are self-sanitized, disinfected and sterilized via a high intensity beam or heat. Examples of door handles that can be sanitized according to this invention include L-shaped door handles 100, U-shaped door handles 200 and knob door handles 300, as shown in FIG. 1A-FIG. 1C, respectively, among others. The self-sanitizing door handles function by delivering a specific type of antimicrobial photodynamic therapy (aPDT), to damage cells with quick lethal effects.

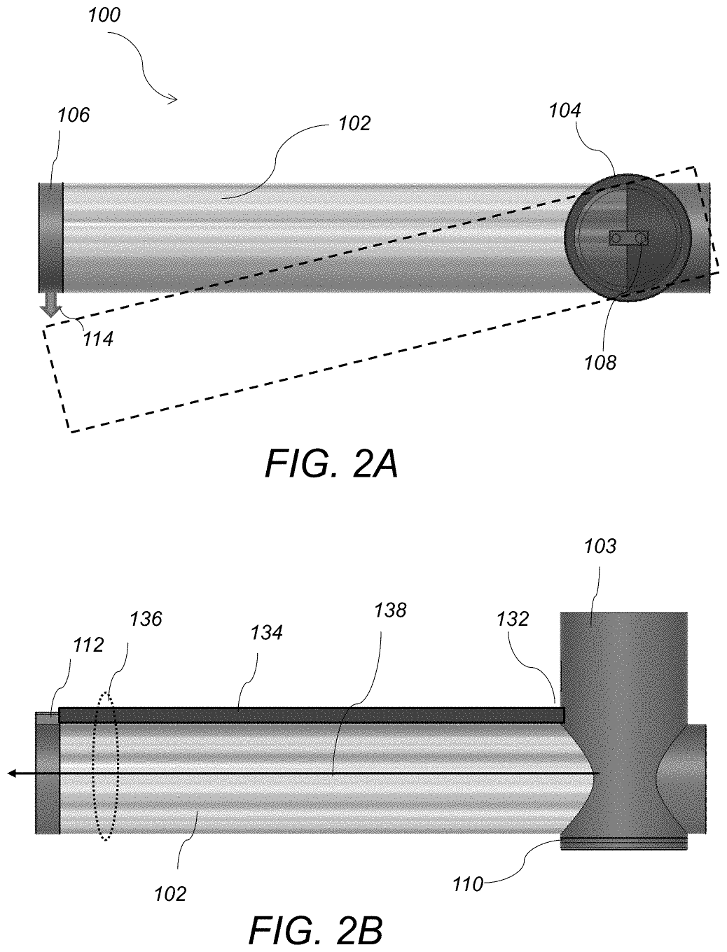

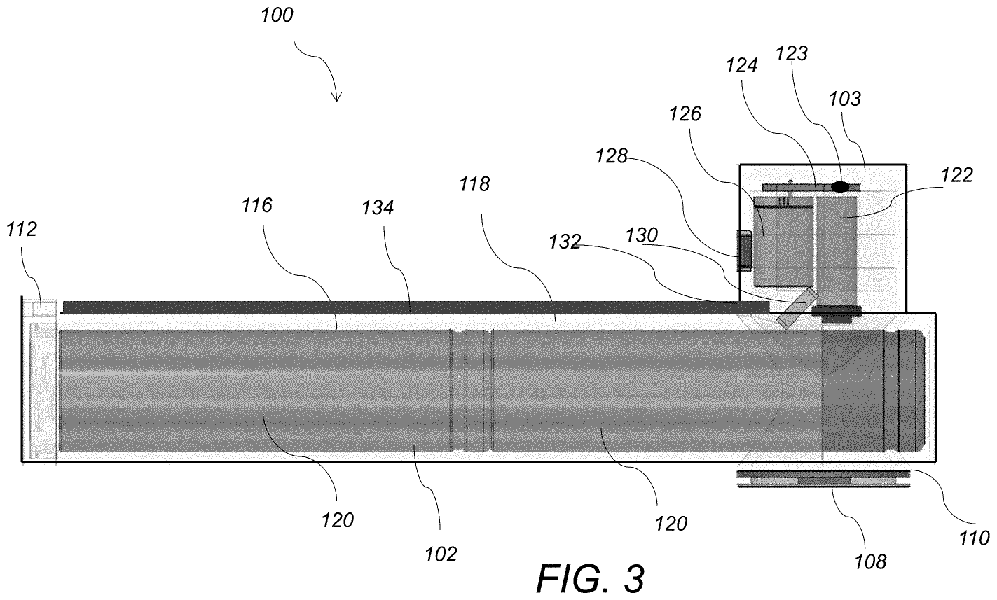

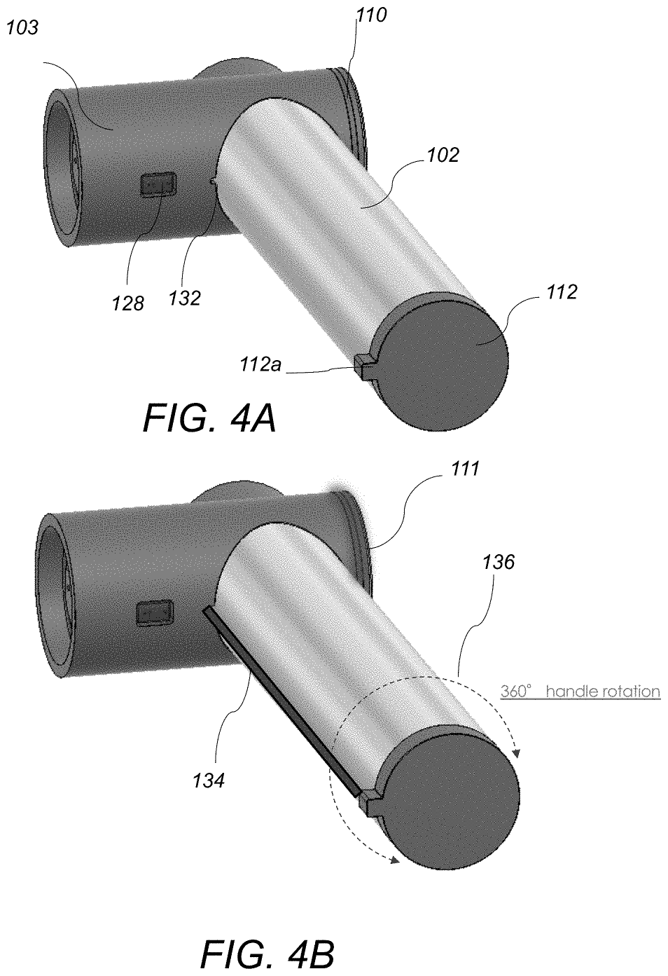

Referring to FIG. 2A-FIG. 4B, a self-sanitizing L-shaped door handle 100 includes a handle bar 102 that has one end attached to a door 70 via a connecting component 103 and one free end. Handle bar 102 is configured to move downward in the direction of arrow 114 in order to actuate the door opening/closing mechanism and open/close the door 70. The front end of connecting component 103 includes a front facing rangefinder 108 and a warning light indicator 110 that is embedded in the rim of component 103. In one example, the rangefinder is a proximity sensor that detects a person within a specified distance from the handle. Examples of proximity sensors include infrared proximity sensors, capacitive proximity sensors, photoelectric proximity sensors and inductive proximity sensors. The warning light indicator is a light emitting diode (LED) light. As shown in FIG. 3, a laser source 126 with a collimator, a laser beam deflector 130, a motor 122 and a circuit board 124 with a programmable processor 123 are incorporated within the interior space of the connecting component 103. A side rangefinder 128 is also included on the inner side of the connecting component 103. Handle bar 102 is a hollow cylinder that includes a static internal shell 116, a rotating outer shell 118 and an end cap 112. End cap 112 includes a laser beam absorber portion 112a. Outer shell 118 is configured to rotate around axis 138 along the circular path 136 by a minimum of 360° degrees. Power to the self-sanitizing mechanism is provided by batteries 120 that are incorporated within the inner space of the static internal shell 116. Upon activation of the self-sanitizing mechanism, a laser beam 134 is emitted from the exit opening 132 of the connecting component 103 and the laser beam is swept along the outer surface of the outer shell 118, while the outer shell 118 is rotated. End cap absorption portion 112a prevents the laser beam 134 from exiting the free end of the handle bar. The sweeping of the outer shell surface with the laser beam and the rotation of the outer shell 118 ensures full laser coverage of the outer surface of shell 118. The laser beam 134 disinfects, sanitizes and sterilizes the outer surface of shell 118 after each activation of the door handle 102. During the disinfecting operation of the laser beam 134 and the available full 360° degrees rotation of the outer shell 118, the warning indication light 110 is lit 111, to indicate the active status of the laser, as shown in FIG. 4B. In one example, laser source 126 is a blue or red or infrared (IR) laser that emits electromagnetic radiation having a wavelength in the range of 400 nm to 850 nm, and has power in the range of 0.1 Watts to 3 Watts, while the outer shell 118 rotates a minimum of 360° degrees within 0.5 to 2.0 sec. In other examples, laser source 126 is an ultraviolet (UV) laser emitting electromagnetic radiation having a wavelength in the range of 180 nm to 400 nm. In addition to the proximity sensors 108 and the visual warning light 110, the system includes additional safety devices that are configured to stop the laser beam when contacted by a person. In one example, the safety device is a conductivity sensor that sends a signal to processor 123 to stop the emission of the laser beam when the handle bar is contacted by human skin. In another example, the safety feature is a shutter that blocks the laser beam when activated by the processor 123.

In operation, a user approaches and opens the door 70 by pressing the handle bar 102 in direction 114. The presence of the user and the user's hand near the handle bar is detected by the front rangefinder 108 and the rear range finder 128 and is communicated to the processor 123 via a first signal. Processor 123 stops the emission of the laser beam 134 and the rotation of the handle bar 102, upon receiving the person detection first signal. When the user subsequently removes his hand from the handle bar 102, the range finders 108, 128 send a second signal to the processor 123 and the processor 123 activates the laser beam sweeping of the outer shell 118 and the rotation of the outer shell 118. The combination of the laser beam sweeping with the rotation of the outer shell ensures that the handle bar is disinfected, sanitized and sterilized after each use.

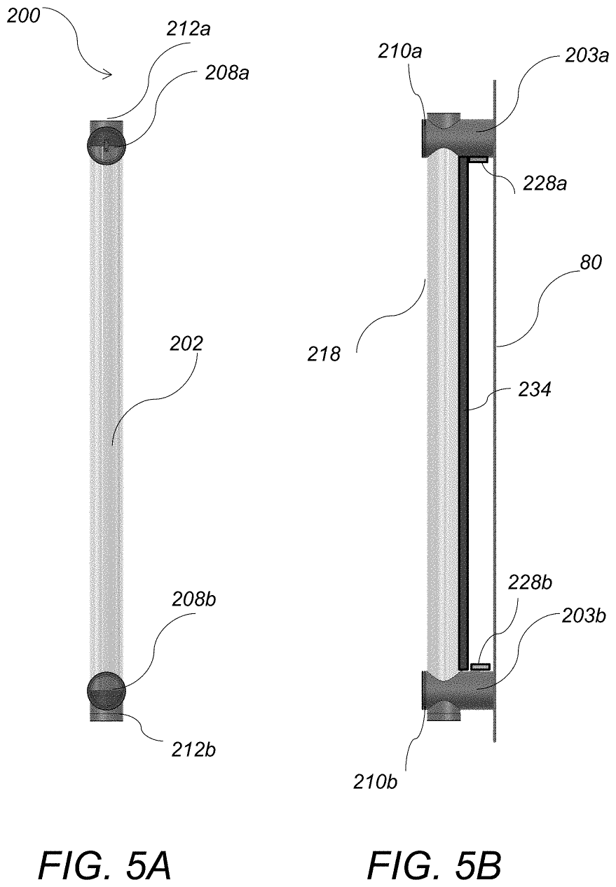

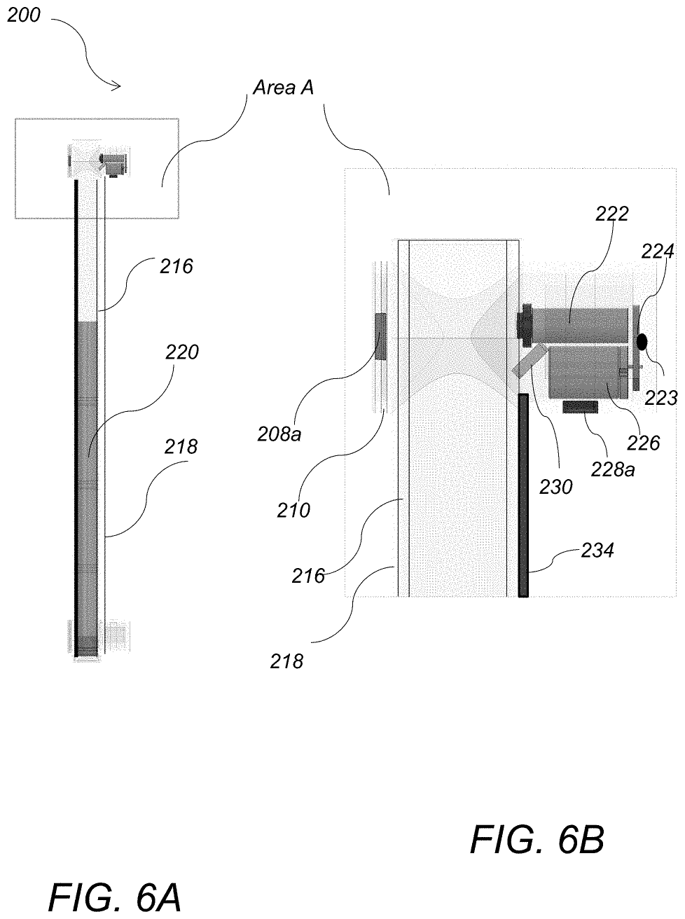

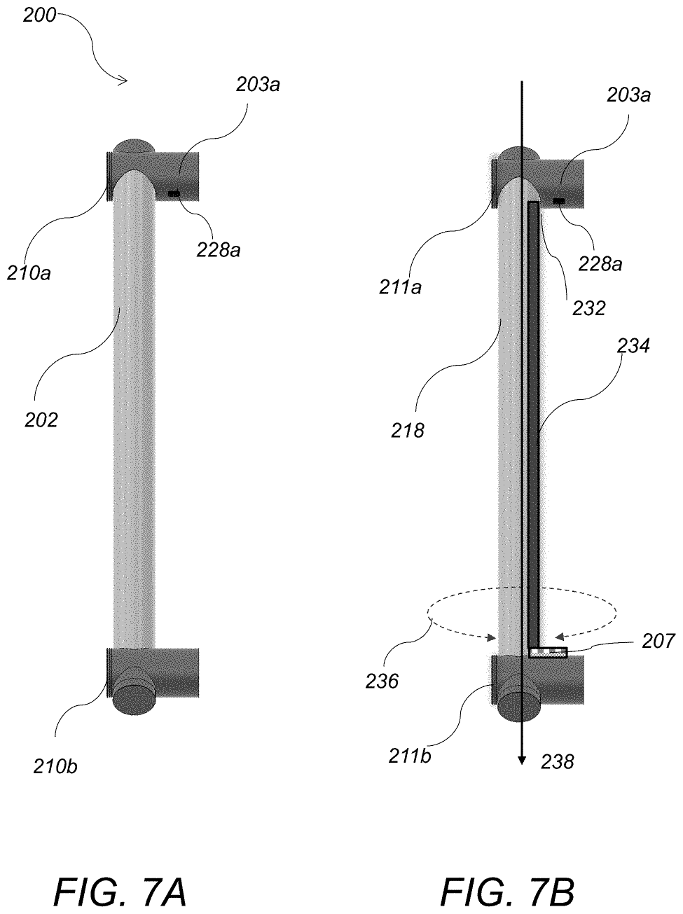

Referring to FIG. 5A-FIG. 7B, a self-sanitizing U-shaped door handle 200 includes a handle bar 202 that has a first end attached to a door 80 via a first connecting component 203a and a second end attached to door 80 via a second connecting component 203b. Handle bar 203 is stationary and is configured to be used to open and close the door 80. The front end of connecting component 203a includes a front facing rangefinder 208a and a warning light indicator 210a, that is embedded in the rim of components 203a. Similarly, the front end of connecting component 203b includes a front facing rangefinder 208b and a warning light indicator 210b, that is embedded in the rim of components 203b. In one example, the rangefinder is a proximity sensor that detects a person within a specified distance from the handle. Examples of proximity sensors include infrared proximity sensors, capacitive proximity sensors, photoelectric proximity sensors and inductive proximity sensors. The warning light indicator is a light emitting diode (LED) light. As shown in FIG. 6A and FIG. 6B, a laser source 226 with a collimator, a laser beam deflector 230, a motor 222 and a circuit board 224 with a programmable processor 223 are incorporated within the interior space of the connecting component 203a. A second rangefinder 228a is also included on the inner bottom side of the connecting component 203a. Similarly, a second rangefinder 228b is also included on the inner upper side of the connecting component 203b. Handle bar 202 is a hollow cylinder that includes a static internal shell 216, a rotating outer shell 218 and top and bottom end caps 212a, 212b. Upper inner side of connecting component 203b also includes a laser beam absorber portion 207. Outer shell 218 is configured to rotate around axis 238 along the circular path 236. Power to the self-sanitizing mechanism is provided by batteries 220 that are incorporated within the inner space of the static internal shell 216. Upon activation of the self-sanitizing mechanism, a laser beam 234 is emitted from the exit opening 232 of the connecting component 203a and the laser beam is swept along the outer surface of the outer shell 218, while the outer shell 218 is rotated. The laser beam absorption portion 207 prevents the laser beam 234 from exiting the second end of the handle bar 202. The sweeping of the outer shell surface with the laser beam and the 360° degrees rotation of the outer shell 218 ensures full laser coverage of the outer surface of shell 218. The laser beam 234 disinfects, sanitizes and sterilizes the outer surface of shell 218 after a user touches the door handle 202. During the disinfecting operation of the laser beam 234 and the 360° degrees rotation of the outer shell 218, the warning indication lights 210a, 210b, are lit 211a, 211b, to indicate the active status of the laser, as shown in FIG. 7B. In one example, laser source 226 emits a wavelength in the range of 450 nm to 850 nm, and has power in the range of 0.1 Watts to 3 Watts, while the outer shell 118 rotates a minimum 360° degrees within 0.5 to 2.0 sec. In other examples, laser source 226 is an ultraviolet (UV) laser emitting electromagnetic radiation having a wavelength in the range of 180 nm to 400 nm. In addition to the proximity sensors 208a, 208b, 228a, 228b and the visual warning lights 210a, 210b, the system includes additional safety devices that are configured to stop the laser beam when contacted by a person. In one example, the safety device is a conductivity sensor that sends a signal to processor 223 to stop the emission of the laser beam when the handle bar is contacted by human skin. In another example, the safety feature is a shutter that blocks the laser beam when activated by the processor 223.

In operation, a user approaches and opens the door 80 by pushing or pulling the handle bar 202. The presence of the user's hand near the handle bar is detected by the front rangefinders 208a, 208b and the rear range finders 228a, 228b, and is communicated to the processor 223 via a first signal. Processor 223 stops the emission of the laser beam 234 and the rotation of the handle bar 202, upon receiving the person detection first signal. When, subsequently, the user removes his hand from the handle bar, the rangefinder detectors 208a, 208b, 228a, 228b send a second signal to the processor 223 that causes the activation of the laser beam sweeping of the outer shell 218 and the rotation of the outer shell 218. The combination of the laser beam sweeping with the rotation of the outer shell ensures that the handle bar is disinfected, sanitized and sterilized after each use.

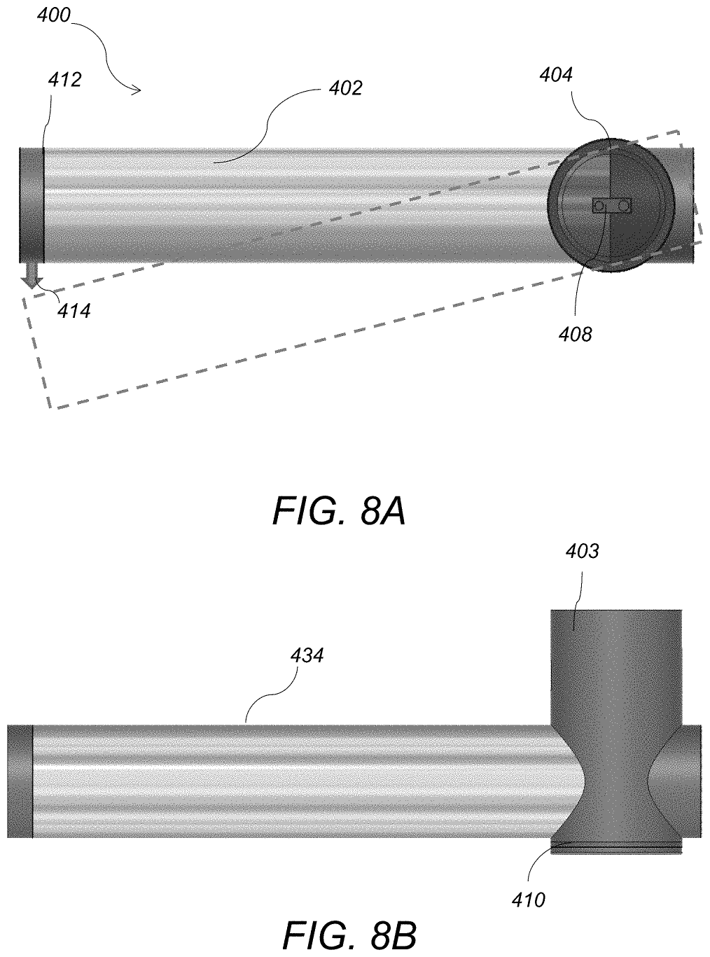

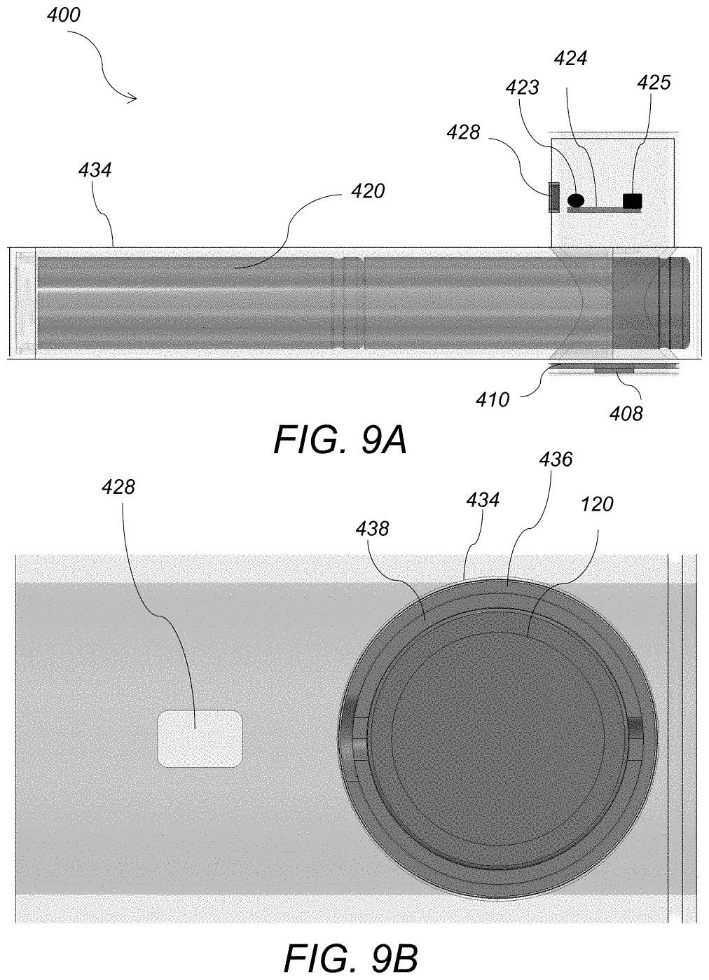

Referring to FIG. 8A-FIG. 9B, an L-shaped door handle 400 that is self-sanitized via a heat flash includes a handle bar 402 that has one end attached to a door 70 via a connecting component 403 and one free end. Handle bar 402 is configured to move downward in the direction of arrow 414 in order to actuate the door opening/closing mechanism and open/close the door 70. The front end of connecting component 403 includes a front facing rangefinder 408 and a warning light indicator 410 is a ring light that is embedded in the rim of component 403. In one example, the rangefinder is a proximity sensor that detects a person within a specified distance from the handle. Examples of proximity sensors include infrared proximity sensors, capacitive proximity sensors, photoelectric proximity sensors and inductive proximity sensors. The warning light indicator is an LED light. As shown in FIG. 9A, a circuit board 424 with a programmable processor 423 and a heating circuit 425 are incorporated within the interior space of the connecting component 403. The heating circuit 425 is configured to generate a heat flash that heats the outer surface of the handle bar 402 to a temperature of above 100° C. in order to sanitize, disinfect and sterilize the handle bar. A side rangefinder 428 is also included on the inner side of the connecting component 403. Handle bar 402 is a hollow cylinder that includes a heat conducting outer shell 434, a primary heat dissipation shell 436, a secondary casing shell 438 and an end cap 412. Outer shell 434 is configured to be heated via the heat flash generated by the heating circuit 425. Power to the self-sanitizing mechanism is provided by batteries 420 that are incorporated within the inner space of the secondary casing shell 438. Upon activation of the self-sanitizing mechanism, the outer shell 434 is heated quickly via a heat flash that kills any microbes, viruses, and bacteria that are present on the door handle outer surface. In one example, the outer shell 434 is a coating made out of a highly conductive material that can be rapidly heated and cooled. The heat flash has a temperature in the range of 80° C. to 195° C. and a duration of 50 msec to 2.5 sec.

In operation, a user approaches and opens the door 70 by pressing the handle bar 402 in direction 414. The presence of the user's hand near the handle bar is detected by the front rangefinder 408 and the rear range finder 428 and is communicated to the processor 423. When the user subsequently removes his hand from the handle bar 402, the range finders 408, 428 send a second signal to the processor 423 and the processor then activates the heat flash generating circuit 425, which then sends a heat flash to heat the outer shell 434 of the door handle. During the flash heating of the door handle, the warning indication light 410 is lit, to indicate the active status of the disinfection process.

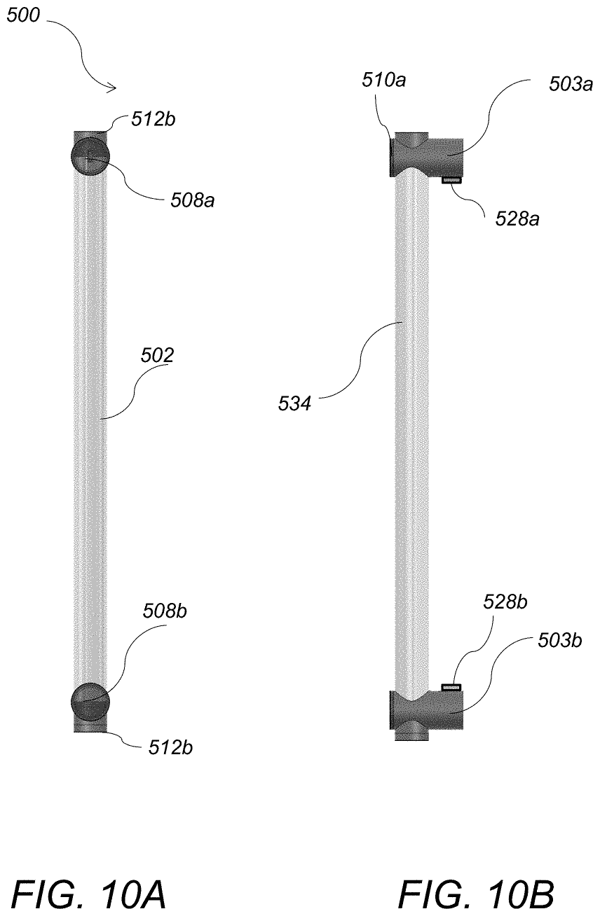

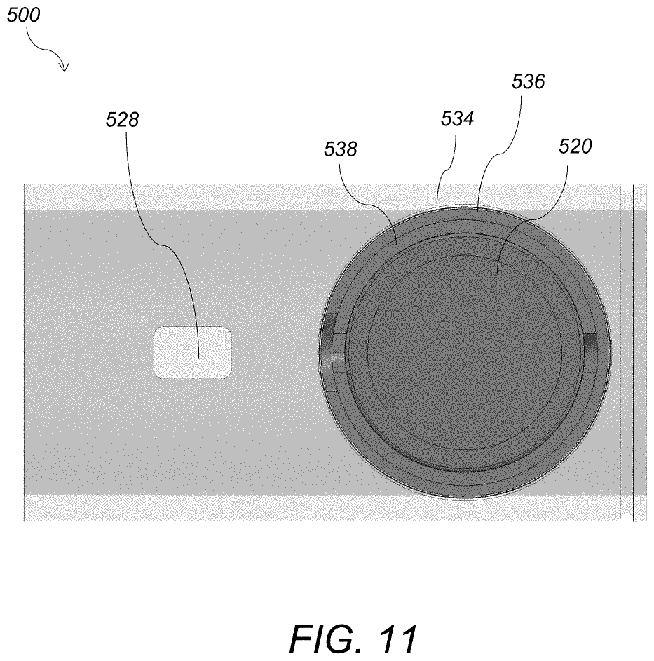

Referring to FIG. 10A-FIG. 11, a U-shaped door handle 500 that is self-sanitized via a heat flash includes a handle bar 502 that has a first end attached to a door 80 via a first connecting component 503a and a second end attached to door 80 via a second connecting component 503b. Handle bar 502 is stationary and is configured to be used to open and close the door 80. The front end of connecting component 503a includes a front facing rangefinder 508a and a warning light indicator 510a. Similarly, the front end of connecting component 503b includes a front facing rangefinder 508b and a warning light indicator 510b. In one example, the rangefinder is a proximity sensor that detects a person within a specified distance from the handle. Examples of proximity sensors include infrared proximity sensors, capacitive proximity sensors, photoelectric proximity sensors and inductive proximity sensors. The warning light indicator is a ring LED light that is embedded in the rim of the component 503a and 503b. As was shown in FIG. 9A, a circuit board 424 with a programmable processor 423 and a heating circuit 425 are incorporated within the interior space of the connecting component 503a. The heating circuit 425 is configured to generate a heat flash that heats the outer surface of the handle bar 502 to a temperature of above 100° C. in order to sanitize, disinfect and sterilize the handle bar. A second rangefinder 528a is also included on the inner bottom side of the connecting component 503a. Similarly, a second rangefinder 528b is also included on the inner top side of the connecting component 503b. Handle bar 502 is a hollow cylinder that includes a heat conducting outer shell 534, a primary heat dissipation shell 536, a secondary casing shell 538 and an end caps 512a, 512b. Outer shell 534 is configured to be heated via the heat flash generated by the heating circuit 425. Power to the self-sanitizing mechanism is provided by batteries 520 that are incorporated within the inner space of the secondary casing shell 538. Upon activation of the self-sanitizing mechanism, the outer shell 534 is heated quickly via a heat flash that kills any microbes, viruses, and bacteria that are present on the door handle outer surface. In one example, the outer shell 534 is a coating made out of a highly conductive material that can be rapidly heated and cooled. Examples, of highly conductive materials include metals, such as nickel, aluminum, chrome, tungsten, copper, silver, gold, zinc, alloys thereof, ceramics such as silicon carbide, graphite and diamond, among others. The heat flash has a temperature in the range of 80° C. to 195° C. and a duration of 50 msec to 2.5 sec.

In operation, a user approaches and opens the door 80 by pushing or pulling the handle bar 502. The presence of the user's hand near the handle bar is detected by the front rangefinders 508a, 508b and the rear range finders 528a, 528b, and is communicated to the processor 423. When, subsequently, the user removes his hand from the handle bar, the rangefinder detectors 508a, 508b, 528a, 528b send a second signal to the processor 423 and the processor then activates the heat flash generating circuit 425, which then sends a heat flash to heat the outer shell 534 of the door handle. During the flash heating of the door handle, the warning indication lights 510a, 510b are lit, to indicate the active status of the disinfection process.

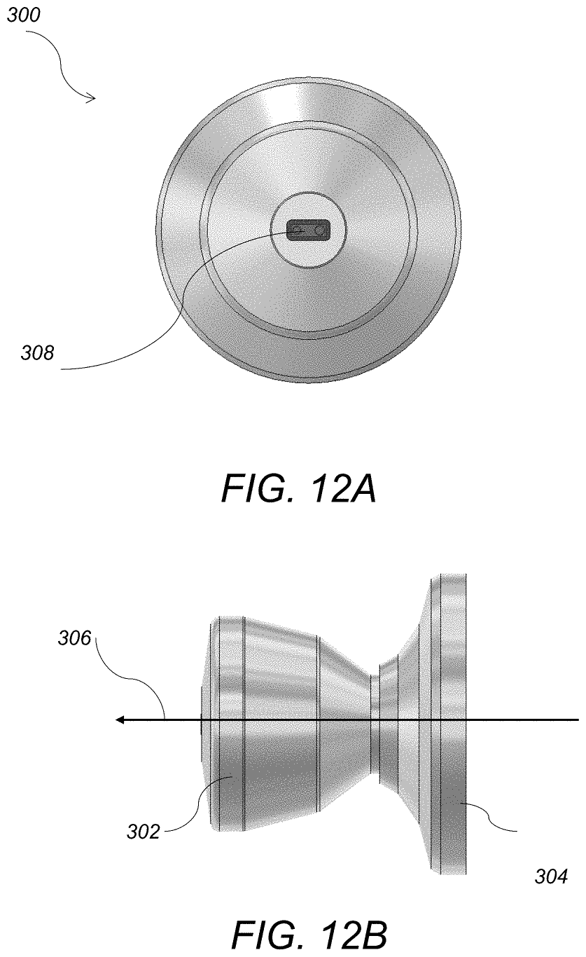

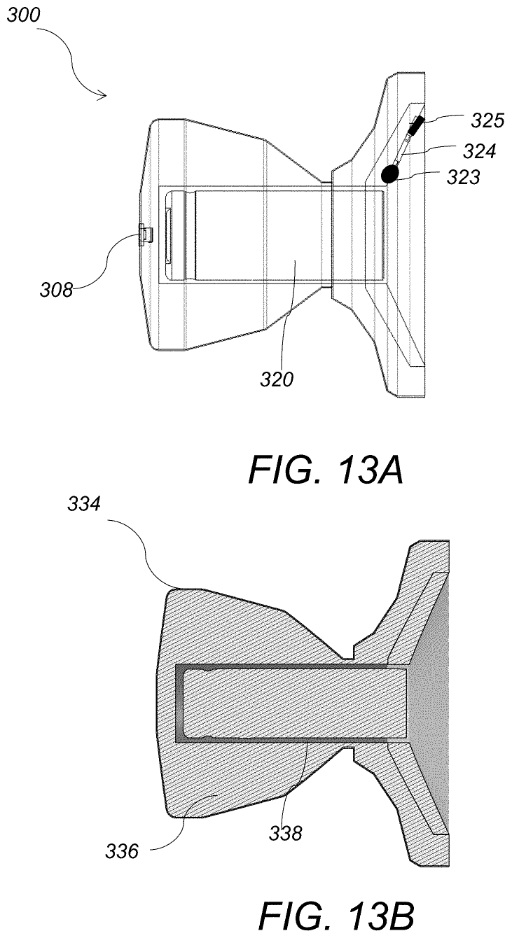

Referring to FIG. 12A-FIG. 13B, a knob-shaped door handle 300 that is self-sanitized via a heat flash includes a rotating knob 302 and a fixed back plate 304 that is configured to be attached to a door 90. Knob 302 is configured to rotate around axis 306 in order to actuate the door opening/closing mechanism and open/close the door 90. The front end of knob 302 includes a front facing rangefinder 308. As shown in FIG. 13A, a circuit board 324 with a programmable processor 323 and a heating circuit 325 are incorporated within the interior space of the back plate 304. The heating circuit 325 is configured to generate a heat flash that heats the outer surface 334 of the knob 302 to a temperature of above 100° C. in order to sanitize, disinfect and sterilize the knob 302. Knob 302 is hollow and includes a heat conducting outer shell 334, a primary heat dissipation shell 336, and a secondary casing shell 338. Outer shell 334 is configured to be heated via the heat flash generated by the heating circuit 325. Power to the self-sanitizing mechanism is provided by batteries 320 that are incorporated within the inner space of the secondary casing shell 338. Upon activation of the self-sanitizing mechanism, the outer shell 334 is heated quickly via a heat flash that kills any microbes, viruses, and bacteria that are present on the knob outer surface. In one example, the outer shell 334 is a coating made out of a highly conductive material that can be rapidly heated and cooled. Examples, of highly conductive materials include metals, such as nickel, aluminum, chrome, tungsten, copper, silver, gold, zinc, alloys thereof, ceramics such as silicon carbide, graphite and diamond, among others. The heat flash has a temperature in the range of 80° C. to 195° C. and a duration of 50 msec to 2.5 sec.

In operation, a user approaches and opens the door 90 by rotating the knob 302. The presence of the user's hand near the knob is detected by the front rangefinder 308 and is communicated to the processor 323. When the user subsequently removes his hand from the knob 302, the range finder 308 sends a second signal to the processor 323 and the processor then activates the heat flash generating circuit 325, which then sends a heat flash to heat the outer shell 334 of the door knob 302.

Other embodiments include one or more of the following. Handle bar 102 of handle 100 is configured to move upwards in the opposite direction of 114. The handle bar 102 may be made of metal, alloys or composite materials. Handles 100, 200, 300, 400, 500 may be retrofitted in existing doors that do not have their own power source. For doors that have an incorporated power source, the power source of the door handle may be eliminated. The laser beam may be an ultraviolet laser. Other high intensity beams that are used include pulsed laser beams, and focused light beams generated by LEDs, gas lamps (xenon, tungsten) or fluorescent lamps. A combination of a laser beam and a heat flash may also be used to disinfect, sanitize and sterilize door handles 100, 200, 300, 400, 500.

Among the advantages of this invention may be one or more of the following. The present invention provides improved sanitization of door handles and prevents the spread of viral, bacterial, and microbial contaminants from door handle surfaces to users. The invention helps reduce Hospital Acquired Infections (HAI's) from door handles and other components that receive enormous use every day by multitudes of people. The door handle sanitization of this invention is anticipated to also work against Multidrug resistance organisms (MDRO's). The present invention also reduces the need to clean and disinfect through direct labor and contact, reduces the use of cleaning chemicals, and improves the working conditions of public buildings and facilities. The self-sanitizing door handles of this invention can be used in hospitals (emergency rooms, patient rooms, doctors offices), public restrooms, schools, museums, government and municipal building, libraries, colleges, banks, portable toilets, and office buildings, among others.

The narrowness and location of the beam source allows, due to the rotation of the handle lever, to greatly limit users from any direct eye exposure to the light.

Several embodiments of the present invention have been described. Nevertheless, it will be understood that various modifications may be made without departing from the spirit and scope of the invention. Accordingly, other embodiments are within the scope of the following claims.

What is claimed is:

1. A system for a self-sanitizing door handle comprising:
a door handle configured to operate opening and closing of a door;
a sensor configured to detect presence of a person in the proximity of the door handle and contact of the door handle by the person;
a device for sanitizing the door handle;
a processor configured to receive a signal from the sensor and activate the device for sanitizing the door handle after being contacted by the person.

2. The system of claim 1, wherein the sensor comprises one of infrared proximity sensors, capacitive proximity sensors, photoelectric proximity sensors or inductive proximity sensors.

3. The system of claim 1, wherein the device for sanitizing the door handle is configured to sanitize the door handle via high intensity electromagnetic radiation, or heat, or combination thereof.

4. The system of claim 1, wherein the device for sanitizing the door handle comprises a laser and the laser emits a high intensity electromagnetic radiation laser beam that sanitizes an outer surface of the door handle.

5. The system of claim 4, wherein the high intensity electromagnetic radiation laser beam comprises one of an infrared (IR) light, ultraviolet (UV) light, or visible light.

6. The system of claim 4, further comprising collimating and reflecting components for directing the laser beam onto the outer surface of the door handle.

7. The system of claim 1, wherein the device for sanitizing the door handle comprises a heating source that generates a heat flash that sanitizes an outer surface of the door handle by applying heat.

8. The system of claim 7, wherein the outer surface of the door handle is heated to a temperature above 100° C. during self-sanitizing.

9. The system of claim 7, wherein the outer surface of the door handle comprises a heat conductive coating.

10. The system of claim 9, wherein the heat conductive coating comprises one of metals, nickel, aluminum, chrome, tungsten, copper, silver, gold, zinc, alloys thereof, ceramics, silicon carbide, graphite or diamond.

11. The system of claim 1, further comprising a motor configured to rotate the door handle while being sanitized.

12. The system of claim 1, further comprising one or more warning indicators that emit one or more warning signals during the sanitizing of the door handle.

13. The system of claim 12, wherein the one or more warning indicators comprise light emitting diodes (LED).

14. The system of claim 1, further comprising a power source that provides power for operating the device for sanitizing the door handle, the sensor and processor.

15. The system of claim 1, further comprising one or more safety interlock devices that are configured to stop the sanitizing of the door handle when the door handle is touched by the person.

16. The system of claim 1, wherein the door handle comprises one of an L-shaped door handle, a U-shaped door handle, or a knob-shaped door handle.

17. The system of claim 1, wherein the sensor, the sanitizing device and the processor are integrated within the door handle.

18. The system of claim 1, wherein the door handle comprises a static internal component and a rotating outer component.

19. A method for self-sanitizing a door handle comprising:
providing a door handle configured to operate opening and closing of a door;
providing a sensor configured to detect presence of a person in the proximity of the door handle and contact of the door handle by the person;
providing a device for sanitizing the door handle;
providing a processor configured to receive a signal from the sensor and activate the device for sanitizing the door handle after being contacted by the person; and
detecting the presence of the person in the proximity of the door handle and contact of the door handle by the person by the sensor and sending a first signal to the processor;
detecting removal of the person's contact of the door handle by the sensor and sending a second signal to the processor;
activating the sanitizing device by the processor and sanitizing the door handle.
